(19) United States
(12) Patent Application Publication
(10) Pub. No.: US 2017/0027868 A1
(43) Pub. Date: Feb. 2, 2017

(54) PEPTIDE-CONJUGATED LIPOSOME

(71) Applicants: Mahmoud Reza Jaafari, Mashhad (IR); Shahrzad Amiridarban, Mashhad (IR); Ali Badiee, Mashhad (IR)

(72) Inventors: Mahmoud Reza Jaafari, Mashhad (IR); Shahrzad Amiridarban, Mashhad (IR); Ali Badiee, Mashhad (IR)

(21) Appl. No.: 15/294,820

(22) Filed: Oct. 17, 2016

Related U.S. Application Data
(60) Provisional application No. 62/272,701, filed on Dec. 30, 2015.

(51) Int. Cl. A61K 9/127; A61K 31/704

(52) U.S. Cl. CPC A61K 9/1271; A61K 31/704; A61K 9/1278

(57) ABSTRACT

Disclosed herein is a liposomal composition for treatment of cancer including PEGylated liposomes loaded by an anticancer drug, where the PEGylated liposome is further targeted by an effective number of P15 peptides.

### CROSS REFERENCE TO RELATED APPLICATION

This application claims the benefit of priority from pending U.S. Provisional Patent Application No. 62/272,701, filed on Dec. 30, 2015, and entitled “TARGETING HDM-2 EXPRESSING CANCER CELLS WITH A NOVEL PEPTIDE AND APPLICATIONS THEREOF IN CANCER TREATMENT,” which is incorporated herein by reference in its entirety.

### TECHNICAL FIELD

The present disclosure generally relates to the field of drug delivery, and particularly to liposome-based drug delivery and more particularly to peptide-conjugated liposomes which target HDM-2 expressing cancer cells and applications thereof in cancer treatment.

### BACKGROUND

For the past three decades, targeting drugs, oligonucleotides, and genes to a specific tissue have been one of the fundamental goals of the pharmaceutical industry. Until recently this has been a very elusive problem. One of the fundamental properties of living cells is their ability to sense and respond to their environment. This is accomplished by a specific set of receptors on the cell surface. Identifying the specific binding proteins and defining their specificity is a big stride towards resolving the problem of specific tissue targeting.

The binding of targeting peptides to drugs may raise many technical problems and in most cases affects the activity of the drug; but encapsulation of drugs in liposomes and other carriers may open new routes to resolve these problems. Encapsulation allows for delivering a large number of drug molecules that are targeted towards a specific tissue, by protecting them from degradation by enzymes. Efficient intracellular delivery of therapeutic agents is one of the major challenges in cancer therapy. In recent years, accumulating evidence has suggested that the use of nano-carriers with targeting agents may be more effective in delivering anti-cancer agents compared to free drugs.

Using targeting agents is particularly important in the case of PEGylated liposomes because polyethylene glycol (PEG) coating on the surface of the carrier reduces drug uptake by cells. However, by using surface ligands and activated endocytosis by receptors, the carrier particle uptake increases in tumor tissue. To date, various ligands including antibodies (or their fragments), small organic molecules, carbohydrates, and peptides have been successfully coupled to various forms of nano-carriers.

Most targeting agents are antibodies (or their fragments). Antibody drug conjugates (ADCs) are equivalent to peptide drug conjugates (PDCs) in terms of potency, but PDCs have better tissue penetration and efficacy in animal and clinical studies. In comparison with the bulk weight or size of mAb (monoclonal antibody) carriers, the peptide carriers may have the advantage of overcoming the interstitial tumor pressure in reaching the tumor interior. The important requirement for using peptides as targeting agent is selective binding of the peptide to the cell surface receptors on the targeted cells. The receptor expression should be higher on the targeted cells than on the non-targeted cells. The peptide should be stable enough in systemic circulation to reach the target cell in an effective concentration.

Since most of the targeting peptides are water-soluble and the phospholipids are oil soluble, specific linkers and several steps are required for binding these two molecules. Well-established chemical reactions have been applied to attach different moieties such as peptides to the lipid or to the surface of preformed liposomes, including amine-carboxylic acid conjugation, disulfide bridge formation, hydrazone bond formation, and the thiol-maleimide addition reaction yielding a thioester bond.

Accomplishing the goal of using targeting peptide may reduce or even eliminate side effects by reducing the amount of drug needed and increasing its effectiveness due to its accumulation in the target tissue. Accordingly, there is a need in the art for a simplified means of incorporating peptides into liposomes for targeting specific cancer cells and delivering anticancer agents to them.

### SUMMARY

The following brief summary is not intended to include all features and aspects of the present application, nor does it imply that the application must include all features and aspects discussed in this summary.

According to a general aspect, the present disclosure describes a liposomal composition for cancer treatment. The composition may include a PEGylated liposome that is loaded with an anticancer drug and may be targeted by an effective number of P15 molecules.

The above general aspect may include the following features. The P15 molecule may be conjugated with distearoylphosphatidylethanolamine-polyethylene glycol-maleimide (DSPE-PEG2000 maleimide). The P15 molecules are P15 peptides with P15 with a sequence of H-Cys-Gly-Gly-Gly-Pro-Pro-Leu-Ser-Gln-Glu-Thr-Phe-Ser-Asp-Leu-Trp-Lys-Leu-Leu-OH. The P15 molecules may bind at least one extracellular domain of an HDM-2 receptor.

In an implementation, the effective number of peptide molecules on the surface of said PEGylated liposome may be between 25 and 200 molecules. According to another implementation, the effective number of peptide molecules on the surface of said PEGylated liposome may be 100.

According to some implementations, the anticancer drug may be selected from Epigallocatechin-3-gallate (EGCG), soy isoflavones, Isoflavones genistein, daidzein, Coumarins, flavonoids, silibinin, polyphenols, baicalin, lycopenes, Vincristine, doxorubicin, cisplatin, 5-fluorouracil, Methotrexate, Cyclophosphamide, mustine, prednisolone, epirubicin, folinic acid, oxaliplatin, etoposide, bleomycin, or combinations thereof.

According to other implementations, the anticancer drug may be selected from doxorubicin, daunorubicin, epirubicin, Idarubicin or derivatives thereof.

In another aspect, the present disclosure describes a method for treating cancer by administering to a patient a therapeutically effective amount of the liposomal composition including a PEGylated liposome that is loaded with an anticancer drug and may be targeted by an effective number of P15 molecules.

### BRIEF DESCRIPTION OF DRAWINGS

While the specification concludes with claims particularly pointing out and distinctly claiming the subject matter that is regarded as forming the present application, it is believed that the application will be better understood from the following description taken in conjunction with the accompanying DRAWINGS, where like reference numerals designate like structural and other elements, in which:

FIG. 1 illustrates a schematic structure of an exemplary P15 peptide-conjugated liposome, consistent with exemplary embodiments of the present disclosure.

FIG. 2 is a flowchart of an example of a method for preparation of P15 peptide-conjugated liposomes, consistent with exemplary embodiments of the present disclosure.

FIG. 3 illustrates high performance liquid chromatography (HPLC) peaks obtained for P15 and DSPE-PEG2000-P15 conjugate as described in detail in connection with example 2, consistent with exemplary embodiments of the present disclosure.

FIG. 4A illustrates the expression of HDM-2 receptor in NIH-3T3 cell line as a negative HDM-2 cells, consistent with exemplary embodiments of the present disclosure.

FIG. 4B illustrate the expression of HDM-2 receptor in C26 cell line as a positive HDM-2 cells, consistent with exemplary embodiments of the present disclosure.

FIG. 5A shows in vitro cellular binding of P15 (100 peptide)-Doxil, Doxil and free doxorubicin to HDM-2-negative NIH-3T3 cells at 4° C., consistent with exemplary embodiments of the present disclosure.

FIG. 5B shows in vitro cellular binding of P15 (100 peptide)-Doxil, Doxil and free doxorubicin to HDM-2-positive C26 cells at 4° C., consistent with exemplary embodiments of the present disclosure.

FIG. 6A shows in vitro cellular uptake of P15 (100 peptide)-Doxil, Doxil and free doxorubicin to HDM-2-negative NIH-3T3 cells at 37° C., consistent with exemplary embodiments of the present disclosure.

FIG. 6B shows in vitro cellular uptake of P15 (100 peptide)-Doxil, Doxil and free doxorubicin to C26 cells at 37° C., consistent with exemplary embodiments of the present disclosure.

FIG. 7A shows percent of changes in animal body in female BALB/c mice bearing C-26 colon carcinoma tumor after intravenous administration of a single dose of 15 mg/kg doxorubicin or dextrose 5% on day 8 after tumor inoculation, consistent with exemplary embodiments of the present disclosure.

FIG. 7B shows average tumor volume in female BALB/c mice bearing C-26 colon carcinoma tumor after intravenous administration of a single dose of 15 mg/kg doxorubicin or dextrose 5% on day 8 after tumor inoculation, consistent with exemplary embodiments of the present disclosure.

FIG. 7C shows survival curve for female BALB/c mice bearing C-26 colon carcinoma tumor after intravenous administration of a single dose of 15 mg/kg doxorubicin or dextrose 5% on day 8 after tumor inoculation, consistent with exemplary embodiments of the present disclosure.

FIG. 8A shows bio-distribution of P15-Doxil formulations, Doxil, and free doxorubicin at different time points (24 and 48 h) in serum, consistent with exemplary embodiments of the present disclosure.

FIG. 8B shows bio-distribution of P15-Doxil formulations, Doxil, and free doxorubicin at different time points (24 and 48 h) in liver and spleen, consistent with exemplary embodiments of the present disclosure.

FIG. 8C shows bio-distribution of P15-Doxil formulations, Doxil, and free doxorubicin at different time points (24 and 48 h) in kidneys, lungs, muscle and heart, consistent with exemplary embodiments of the present disclosure.

FIG. 8D shows bio-distribution of P15-Doxil formulations, Doxil, and free doxorubicin at different time points (24 and 48 h) in tumor in BALB/c mice bearing C-26 tumor after a single dose of 15 mg/kg liposomal doxorubicin administered two weeks after the tumor inoculation, consistent with exemplary embodiments of the present disclosure.

### DETAILED DESCRIPTION

Cytotoxic drugs that are used for the treatment of cancer should be delivered inside the cancerous cells in order to exert their therapeutic effect. However, cell membranes prevent therapeutic molecules from entering the cells, which is considered a major challenge in drug delivery to cells. That is particularly challenging in case of drug carriers, such as PEGylated liposomes. PEGylated liposomes, are liposomes with polyethylene glycol (PEG) chains attached thereto. PEG chains on the surface of PEGylated liposomes may reduce drug uptake by the cells. As used in this disclosure, liposomes are structures, which consist of at least one lipid bilayer surrounding an aqueous core. The liposomes can accommodate a number of drugs of varying lipophilicity, such as cytotoxic anticancer drugs (e.g., doxorubicin, paclitaxel, etc.)

Selective targeting of a therapeutic agent, such as cytotoxic anti-cancer drugs may increase the effective amount of the drug delivered to a cell or tissue while reducing the likelihood that the compound will have an adverse effect on other non-targeted cells, tissues or organs.

Disclosed herein is an exemplary PEGylated liposomal composition containing a peptide as a targeting ligand which may be used for targeting a therapeutic agent to a cell that is expressing a particular surface receptor. The selective targeting may be achieved as a result of selective binding between the peptide and the surface receptors on the targeted cells. The receptor expression should be higher on the targeted cells than on the non-targeted cells. Moreover, the PEGylated liposomal compositions containing a peptide have the advantage of overcoming the interstitial tumor cell pressure and thereby reach the tumor cell interior.

The PEGylated composition described in this disclosure utilizes P15 peptide as the targeting ligand. P15 peptide has shown potent binding affinity to the cell surface HDM-2 receptor. The level of HDM-2 expression is high in the membrane of a variety of cancer cells (such as colon carcinoma, melanoma, pancreas and breast cancer) but not normal cells. Therefore, P15 peptide engrafted liposomes target HDM-2 receptors on tumor cells and PEGylated liposomal compositions containing P15 peptide as the targeting agent (P15-liposomes) may be utilized to deliver anticancer drugs (e.g., doxorubicin, paclitaxel, etc.) to HDM-2 expressing cancer cells.

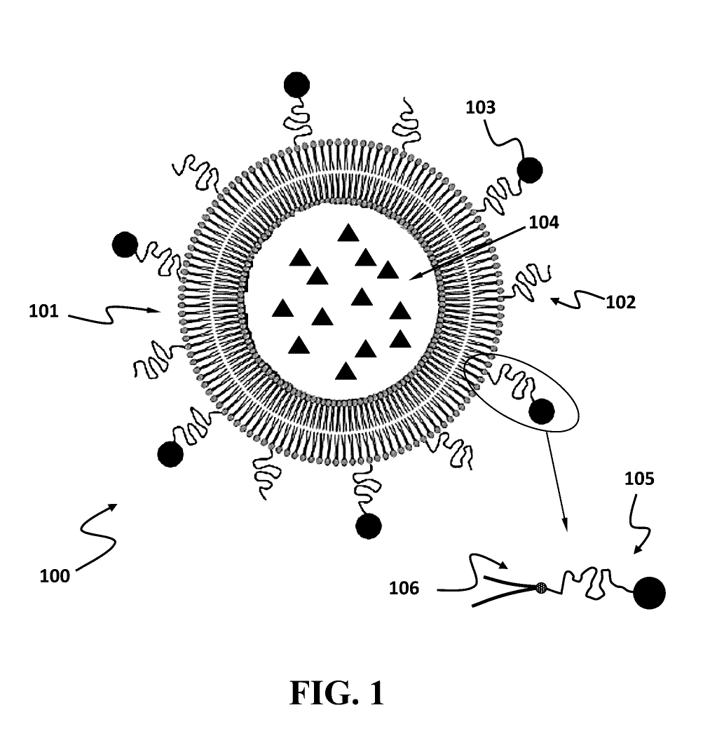

FIG. 1A illustrates a schematic representation of an exemplary P15-liposome 100, consistent with exemplary embodiments of the present disclosure. P15-liposome 100 is a P15 peptide-conjugated liposome which includes liposome 101, PEG linkers 102, and P15 peptide molecules 103. An anticancer drug 104, such as Doxorubicin, Daunorubicin, Epirubicin, Valrubicin, and Idarubicin may be encapsulated in the liposome 101. In an exemplary implementation, first a phospholipid-PEG may be conjugated with a P15 peptide to prepare phospholipid-PEG-P15 105, and then the phospholipid-PEG-P15 is used in targeting preformed liposome 101.

In an implementation, the anticancer drug may include, but is not limited to Epigallocatechin-3-gallate (EGCG), soy isoflavones, Isoflavones genistein, daidzein, Coumarins, flavonoids, silibinin, polyphenols, baicalin, lycopenes, Vincristine, doxorubicin, cisplatin, 5-fluorouracil, Methotrexate, Cyclophosphamide, mustine, prednisolone, epirubicin, folinic acid, oxaliplatin, etoposide, bleomycin, and combinations thereof.

PEG linkers are capable of linking a peptide to the liposome. As used herein, “PEG” refers to polyethylene glycol. As used herein, the terms “polyethylene glycol” and “PEG” broadly encompass any polyethylene glycol molecule known in the art. Polyethylene glycol is typically a water-soluble polymer. The number of ethylene glycol units in PEG is approximated for the molecular mass described in Daltons. For example, if two PEG molecules are attached to a peptide where each PEG molecule has the same molecular mass of 10 kDa, then the total molecular mass of PEG on the peptide is about 20 kDa. The molecular masses of the PEG attached to the peptide can also be different, e.g., of two molecules on a peptide one PEG molecule can be 5 kDa and one PEG molecule can be 15 kDa. It is well known in the art that a PEG linking molecule can be lengthened to a desired length by one of ordinary skill in the art by adding additional PEG molecule together. In a PEGylated liposome, the PEG linkers are linked to the liposome prior to linking the PEG linkers to the peptide. Referring to FIG. 1 following the linkage of the peptide 103 to the liposome 101 via the PEG linker 102, liposome-PEG-peptide 105 is produced.

P15 peptide 103 is a domain of P53 protein and has a sequence of H-Cys-Gly-Gly-Gly-Pro-Pro-Leu-Ser-Gln-Glu-Thr-Phe-Ser-Asp-Leu-Trp-Lys-Leu-Leu-OH which may bind to at least one of the extracellular domains of HDM-2 receptor.

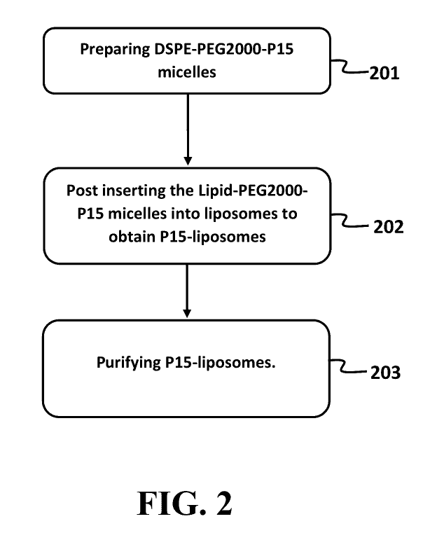

The P15-liposome formulations of exemplary embodiments of the present disclosure, which are P15 peptide-conjugated PEGylated liposomes, may be prepared by a post insertion method. FIG. 2 is a flowchart of an example of a method for preparation of P15-liposomes, consistent with exemplary embodiments of the present disclosure.

Referring to FIG. 2, preparation of P15-conjugated liposomes may include preparing distearoylphosphatidylethanolamine-polyethylene glycol-maleimide (DSPE-PEG2000)-P15 micelles (step 201), post inserting the Lipid-PEG2000-P15 micelles into liposomes to obtain P15-liposomes (step 201), and purifying P15-liposomes (step 203).

Referring to step 201, in an exemplary implementation, in order to prepare DSPE-PEG2000-P15 micelles, first, the P15 peptide may be conjugated with DSPE-PEG2000 maleimide, after conjugation, the organic solvents may be removed to obtain a lipid film, and the lipid film may then be hydrated to form micelles.

In an implementation, the P15 peptide may be conjugated with DSPE-PEG2000 maleimide by first, dissolving the P15 peptide in an organic solvent, such as dimethyl sulfoxide (DMSO) in order to prepare a peptide solution and dissolving DSPE-PEG2000 maleimide in an organic solvent, such as chloroform, diethyl ether and ethanol. Then, the peptide solution may be mixed with DSPE-PEG2000 maleimide solution with a specific peptide:phospholipid molar ratio under an inert environment. The mixture may be incubated at room temperature in the dark, while being stirred overnight. In an implementation, the P15 peptide solution may be mixed with phospholipid solution at a 1:1 (volume/volume) ratio. Peptide may be reacted with DSPE-PEG2000 maleimide phospholipid in a molar ratio of about 1.2:1 (P15 peptide:phospholipid) under inert atmosphere like argon.

In step 201, after conjugating the peptide with DSPE-PEG2000 maleimide, the organic solvents may be removed to obtain a lipid film. Based on the solvent used, different methods may be utilized to remove the solvent. According to some implementations, chloroform may be removed by rotary vacuum evaporation and DMSO may be removed via freeze drying. Furthermore, solvent traces may be removed under an inert environment. Once the solvents are removed a lipid film is obtained. After that, the lipid film may be hydrated with injectable water in order to obtain micellized DSPE-PEG2000-P15.

In step 202, post insertion may be carried out by co-incubation of DSPE-PEG2000-P15 micelles obtained in step 101 and liposomes at the transition temperature of the lipid bilayer, which may be, for example, 55 to 60° C. The co-incubation may be carried out for about 1 to 4 hours under gentle shaking in order to obtain P15-targeted liposomes. Certain amounts of the micelles may be used to produce different numbers of P15 ligands on each liposome. Different targeted liposomes with different numbers of ligands may be produced. In an implementation, the number of P15 ligands on each liposome may include 25, 50, 100, and 200.

In step 203, P15-liposomes are purified to separate the P15-targeted liposomes from PEG micelles and free peptides. To this end, in an exemplary implementation, dialysis technique may be used against Histidin buffer with 10 mM concentration at 6.5 pH and with 30 kDa molecular weight cut off dialysis membrane for four times at 4° C.

### EXAMPLES

### Example 1

### Preparation of P15 Peptide-Conjugated Liposomes

In this example, P15-liposomes were prepared with the following steps, according to one or more aspects of the present disclosure. In a first step, DSPE-PEG2000-P15 micelles were prepared. The preparation of P15 peptide-conjugated DSPE-PEG2000-maleimide [DSPE-PEG2000-P15] micelles may be carried out by preparing P15 peptide solution by dissolving it in the dimethyl sulfoxide (DMSO) and preparing a phospholipid solution by dissolving maleimide-PEG2000-DSPE in chloroform.

After that, the peptide and lipid solution were mixed with each other at 1:1 concentration ratio (volume/volume). The peptide reacted with maleimide-PEG2000-DSPE in a molar ratio of 1.2:1 under argon atmosphere and incubated at room temperature in dark with magnet stirring overnight.

In the next step, in order to form the lipid film, the organic solvents were removed by rotary vacuum evaporation (for chloroform) and freeze drier (for DMSO); also solvent traces were removed under nitrogen gas. The lipid film was then hydrated with injectable water for the formation of DSPE-PEG2000-P15 micelles.

After that, DSPE-PEG2000-P15 micelles were post-inserted to liposomes for obtaining P15 conjugated liposomes. To optimize the best peptide density on the surface of each liposome, four types of peptide-conjugated liposomes were prepared by controlling the number of P15 peptides on the vesicle; so post insertion of DSPE-PEG2000-P15 micelles into the liposomes (DOXIL®) was carried out using certain amounts of the micelles in order to create 25, 50, 100 and 200 ligands on each liposome.

In order to prepare different types of P15-liposomes, at first the number of liposomes in 1 ml of Doxil was calculated by considering phospholipid concentration of Doxil which is 13.272 mole/liter and based on the fact that a liposome with 100 nm diameter has about 80,000 phospholipid molecules. Then considering that each phospholipid in micelles was conjugated to a P15 peptide (100% linking efficacy), the exact amount of P15 peptide in DSPE-PEG2000-P15 micelles was indirectly determined by measuring total amount of phospholipid in DSPE-PEG2000-P15 micelles by using a phosphorus assay method.

After that, the molar ratio of 1:7.8 for DSPE-PEG2000-P15 micelle lipid/Doxil lipid and in volumes of 2.5, 4.9, 9.8, and 19.5 micro liter for different types of P15-Doxil (25, 50, 100 and 200 ligands) was determined.

Based on the above explanation, P15-PEG2000-DSPE micelles were subsequently incorporated into liposomal doxorubicin (Doxil) at molar ratio of 1:7.8 (DSPE-PEG2000-P15 micelle lipid/Doxil lipid) and in volumes of 2.5, 4.9, 9.8, and 19.5 micro liter for different types of P15-Doxil (25, 50, 100 and 200 ligands/liposome) respectively. Then the micelle-liposome mixtures were co-incubated at 60° C. for 4 hours with gentle shaking for obtaining P15-liposomes.

Finally, purification of P15-liposomes was carried out by separating the targeted liposomes from PEG micelles and free-peptide liposomes by using dialysis technique against Histidin buffer in sucrose 10% (Histidin 10 mM, pH 6.5) with 30 kDa molecular weight cut off (MWCO) dialysis membrane (Spectrum) four times at 4° C.

### Example 2

### Characterization of P15 Peptide-Conjugated Liposomes

There are some indications which could be assessed regarding to liposomal formulations. In this example, size distribution, polydispersity index and zeta potential of four different types of P15-liposomes were measured by using a dynamic light scattering particle size analyzer instrument (Nano-ZS; Malvern, UK). The mean size of hydrodynamic particle was represented as the value of Z-average size.

The particle diameter of each sample, together with its polydispersity index, was measured using a Dynamic Light Scattering Instrument. The zeta potential of liposomes was determined on the same machine using the zeta potential mode. The physical properties of liposomes containing Dox and its properties after conjugating to different numbers of P15 peptide molecules are presented in Table 1.

Prepared liposomal formulations had a diameter around 100 nm, with variances between about 96 to about 102 nm. Overall, the formulations were very homogenous and had a polydispersity index of <0.3. Zeta potential of prepared liposomes ranged between −18 (for Doxil) to −19.1 (for P15 (200 ligand)-Doxil), as shown in TABLE 1.

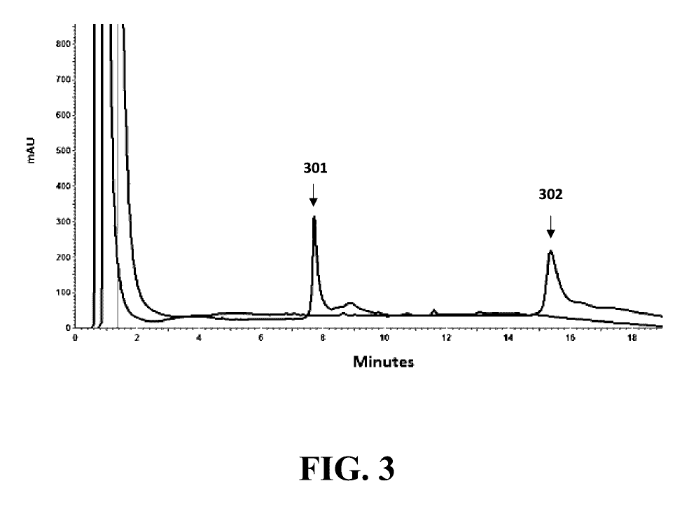

Also the efficiency of peptide linkage was determined using high performance liquid chromatography (HPLC). Briefly, a C18 column (Shiseido Co, Ltd, Tokyo, Japan) was used with a mobile phase of 0.1% trifluoroacetic acid in water (eluent A) and 0.1% trifluoroacetic acid in acetonitrile (eluent B). The eluent gradient increased from 35% A to 60% B in 10 minutes. Referring to FIG. 3, at first free P15 was dissolved in dimethyl sulfoxide (DMSO) and then analyzed by HPLC, so a related peak 301 was separated with a retention time of 8 minutes at 220 nm. Then DSPE-PEG2000-P15 conjugate analyzed by HPLC and the only observed peak 302 was related to DSPE-PEG2000-P15 conjugate with a retention time of about 16 min at 220 nm; so absence of free P15 peptide peak 301 in HPLC analysis of DSPE-PEG2000-P15 conjugate, confirmed that the peptide linkage efficacy was 100%.

Evaluation of the post-insertion method was also done as a leakage stability of liposomes by measuring doxorubicin concentrations in the fractions of eluent before and after post insertion through measuring fluorescence at λEx/Em=480/590 nm using a spectro-fluorometer (Spectra Max M5, Molecular Devices). Aliquots of preparations (Doxil and P15-Doxil) were dissolved in acidified isopropyl alcohol and concentration of Dox before and after post insertion was measured using serial dilution of Doxil through dispersion in RPMI:FCS (70:30 v/v, pH 7.4). The percent of Dox encapsulated was determined by the following formula: % Dox loading efficacy=(Dox concentration after post insertion/Dox concentration before post insertion)×100. Table 1 shows the results of doxorubicin loading efficacy as the evaluation of post insertion method.

TABLE 1
Characteristics of P15-Doxil and non-targeted Doxil.

| | | | | Dox |
|---|---|---|---|---|
| | | Poly- | Zeta | loading |
| | Z-Average | dispersity | potential | efficacy |
| Liposomes | (nm) | index | (mv) | (%) |
| P15(25 ligand)- | 98.1 ± 2.1 | 0.214 ± 0.013 | −18 ± 0.3 | 97 ± 2.3 |
| Doxil | | | | |
| P15(50 ligand)- | 99.3 ± 1.6 | 0.189 ± 0.017 | −18.5 ± 0.1 | 96 ± 3.5 |
| Doxil | | | | |
| P15(100 ligand)- | 99.8 ± 2.5 | 0.231 ± 0.024 | −18.8 ± 0.3 | 94 ± 5.2 |
| Doxil | | | | |
| P15(200 ligand)- | 100 ± 2.8 | 0.221 ± 0.021 | −19.1 ± 0.2 | 95 ± 4.1 |
| Doxil | | | | |
| Doxil | 98 ± 0.9 | 0.121 ± 0.011 | −18 ± 0.1 | 98 ± 2.1 |

### Example 3

### Investigation of HDM-2 Overexpression on C26 and NIH-3T3 Cells

In this example, the overexpression of HDM-2 protein on C26 (colon carcinoma) and NIH-3T3 (mouse embryonic fibroblast) cell lines were evaluated by flow cytometry. The C26 cells were cultured in RPMI 1640 medium and NIH-3T3 cells were cultured in Dulbecco's Modified Eagle's medium (DMEM). Both media were supplemented with 10% (volume/volume) heat-inactivated FCS (Fatal Calf Serum), 2 mM L-glutamine, as well as 100 IU/ml penicillin and 100 mg/ml streptomycin; after that the cells were incubated at 37° C. with a 5% CO2/95% air humidified atmosphere.

The cells were re-suspended in staining buffer (2% FCS and 2 mM EDTA in PBS), then approximately 10⁶ cells were transferred to the flow cytometry tubes and centrifuged for 6 minutes at 4° C. (1500 rpm). After that, the supernatant was disposed and 1 μg of the primary MDM2 antibody (a mouse monoclonal IgG1) was added in remaining buffer (approximately 50 μl) and incubated at 4° C. for 45 minutes in dark. The cells were washed twice with staining buffer to remove not sticking antibodies. Then, the secondary antibody which consists a stain (goat anti-mouse IgG1-FITC) was added and incubated at 4° C. for 30 minutes in dark. After that cells were washed twice with staining buffer.

Then the cells were re-suspended in 1 ml staining buffer to analyze fluorescent-activated cell sorting (FACS) (BD Biosciences, San Jose, USA). All steps except adding secondary antibody, were done for unstained cells as control tubes. Cells were incubated with FITC-labeled anti-HDM-2 antibodies. Antibodies were provided from Santa Cruz Biotechnology, USA.

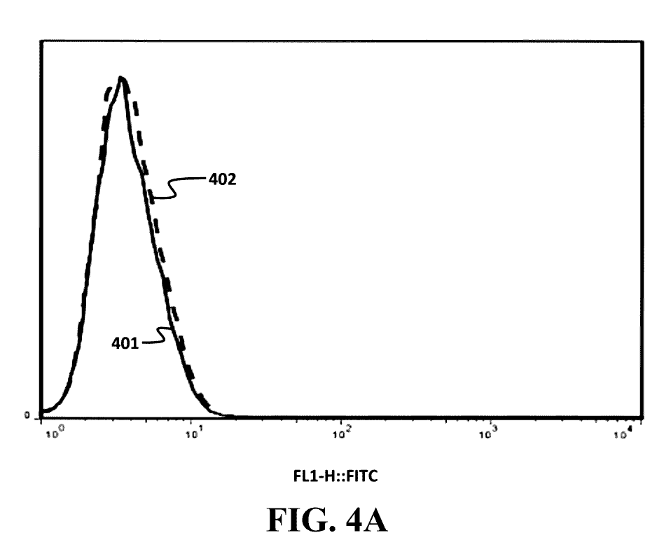

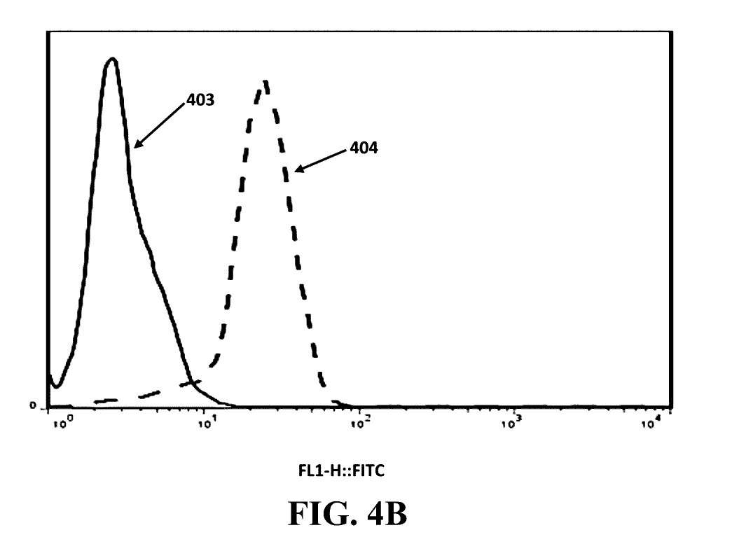

Referring to FIGS. 4A and 4B, the expression of HDM-2 receptor in NIH-3T3 cell line (A) and C26 cell line (B) are determined by FACS analysis. FIG. 4A shows that stained NIH-3T3 cells graph (dashed line designated as 402) is the same as unstained NIH-3T3 cells graph as control group (solid line designated as 401); therefore it shows that NIH-3T3 cell line is a negative HDM-2 cell line and HDM-2 receptor is not overexpressed on it; but in FIG. 4B it is shown that stained C26 cell graph (solid line designated as 404) is different (p<0.001) from unstained C26 cells graph as control group (dashed line designated as 403); so it can be understood that C26 cell line is a positive HDM-2 and HDM-2 receptor is overexpressed on this cell line.

### Example 4

### MTT Cell Proliferation Assay

In this example, the anti-proliferative effects of prepared P15-liposomes containing doxorubicin were assessed on C26 cells by performing MTT assay. At first, C26 cells were seeded in RPMI 1640 media in 96-well plates (5000 cells/well) and were incubated at 37° C. After overnight incubation, cells were treated with different concentration of drug (1:2 serial dilutions of liposomal doxorubicin or free doxorubicin in FCS-free medium) and then incubated at 37° C. for 3 and 6 hours.

After scheduled incubation times, cells were washed with pre-warmed complete culture media and re-incubated further for 48 hours at 37° C. and then the viability of cells was determined by MTT assay; for this purpose, 20 ml of MTT (Thiazolyl Blue Tetrazolium Bromide; Sigma-Aldrich) was added to each well of the plate and was incubated for 4 hours at 37° C.

Then the medium was replaced with 200 μl of Dimethyl sulfoxide (DMSO) for 10 min and the absorbance was determined at 550 nm by using a Multiskan plus plate and In vitro cytotoxicity effect (IC50) values were calculated using CalcuSyn version 2software (BIOSOFT, UK). Relative cell death was calculated as follows: R=(A control−A test)/(A control−A blank) where A test and A control were the absorbance of the cells treated with the test dilutions and the culture medium (negative control). A blank was the absorbance of cell free wells.

The concepts of IC50 is the drug concentration causing 50% inhibition of biological activity of cancer cells; so referring to Table 2, IC50 of Doxil and P15-Doxil were lower than free Doxorubicin. Also the IC50 of P15-Doxil formulations were lower than non-targeted Doxil which means higher cytotoxicity of targeted formulations (P15 Doxil) compared to Doxil formulation.

TABLE 2
In vitro cytotoxicity effect (IC50) of P15-Doxil, Doxil, and free
doxorubicin treatment on C-26 cells after different exposure times.

| | | IC50 (μM ± SD) | |
|---|---|---|---|
| Cell line | Treatment | 3 h | 6 h |
| C26 | Doxil | 8.144 ± 0.05 | 5.043 ± 0.21 |
| | P15 (25pep.)-Doxil | 8.500 ± 0.08 | 5.007 ± 0.53 |
| | P15 (50pep.)- Doxil | 7.351 ± 0.12 | 4.078 ± 0.63 |
| | P15 (100pep.)- Doxil | 5.437 ± 0.26 | 2.975 ± 1.23 |
| | P15 (200pep.)- Doxil | 3.129 ± 0.06 | 2.475 ± 2.11 |
| | Free doxorubicin | 0.625 ± 0.07 | 0.302 ± 0.05 |

### Example 5

### Evaluation of P15-Specific Cellular Binding and Uptake

Since C26 cells overexpress HDM-2 receptor on their surfaces, and the P15 used in this study, having a binding domain for HDM-2 receptors, the binding affinity of P15 peptide to C26 cells (HDM-2-positive) was compared to NIH3T3 cells (HDM-2-negative) which were used to assess any non-specific interactions. The exemplary procedure was as follows: cells were detached from cell culture dishes by using 0.05% trypsin and 0.02% EDTA (GIBCO). Approximately 10⁶ cells were re-suspended in staining buffer, fluorescein isothiocyanate (FITC) in PBS containing 2% FCS and 2 Mm EDTA, for 1 hour at 4° C. in darkness; and then incubated with free doxorubicin, liposomal doxorubicin (Doxil) and p15-Doxil (20 μg doxorubicin/ml) at 4° C. for 1 hour. Afterward, the cells were washed three times with PBS and re-suspended in an appropriate volume of the buffer and were analyzed by flow cytometry. BD FACS Calibur equipped was used to read fluorescence signal.

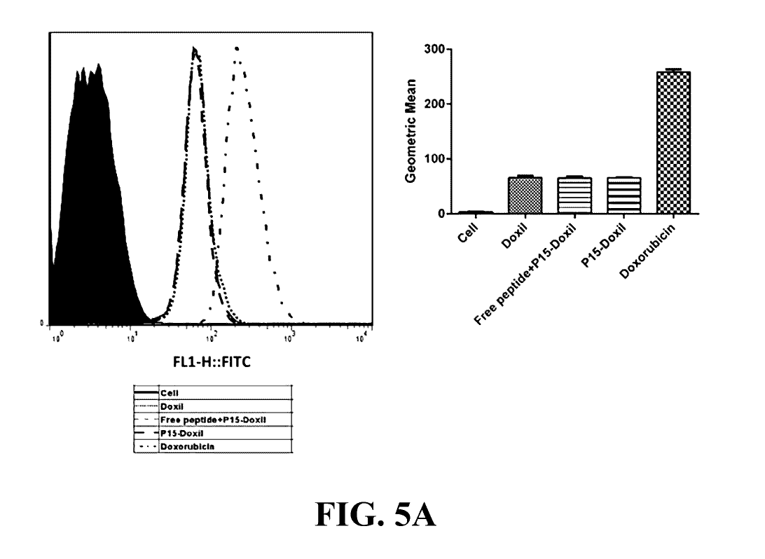

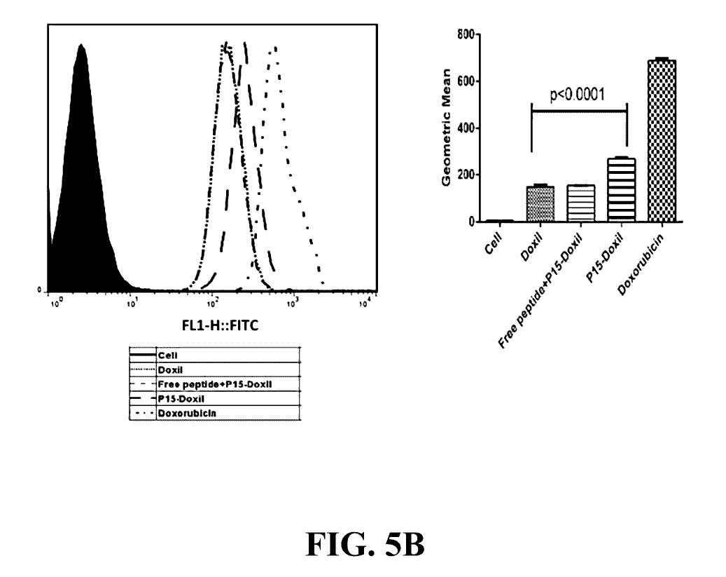

Referring to FIGS. 5A and 5B, the binding of doxorubicin in both C26 and NIH3T3 cells is more than other forms of drugs, because free doxorubicin may enter the cells by simple diffusion. Also binding of Doxil is the same in both C26 and NIH3T3 cells, because it is not targeted but binding of P15 Doxil is more in C26 cells than NIH3T3 cells and it may be because of overexpression of HDM-2 receptors in C26 cells. The specificity of P15-Doxil binding was also evaluated by a competitive assay so cells were pre-incubated for 30 minutes with excess amounts of free P15 peptide, before introducing P15-Doxil, which could competitively inhibit the binding of P15-Doxil. Therefore, the results of free peptide+P15 Doxil experiment should be the same as Doxil experiment in both C26 and NIH3T3 cells.

For evaluating in vitro cellular uptake, NIH3T3 cells were seeded in complete DMEM and C26 were seeded in complete RPMI in 24-well plates (2×10⁵ cells/well). The cells were incubated overnight and then they were treated with free doxorubicin, Doxil and P15-Doxil (20 μg doxorubicin/ml,) for 3 hours in 1 ml serum-free media at 37° C. Then, cells were washed with PBS, detached by 100 μl of trypsin-EDTA solution (Gibco, UK) and centrifuged at 1500 rpm for 5 minutes. The cells were washed with cold PBS three times and resuspended in staining buffer before analysis. The competitive assay was performed to confirm the specific uptake of P15-Doxil in vitro. Then 0.9 ml of an acidified isopropanol was added to each well, and the mixture was incubated overnight at 4° C. to extract the cell-associated doxorubicin. After sedimentation of the cell debris, doxorubicin concentration was measured.

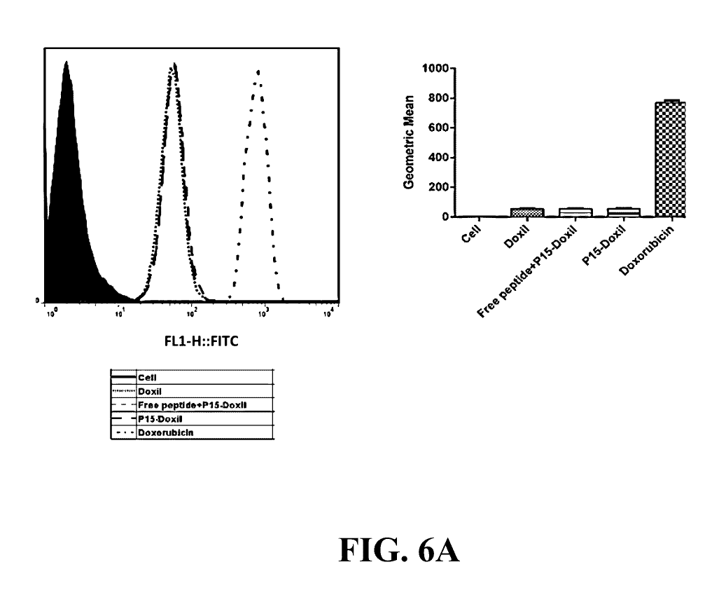

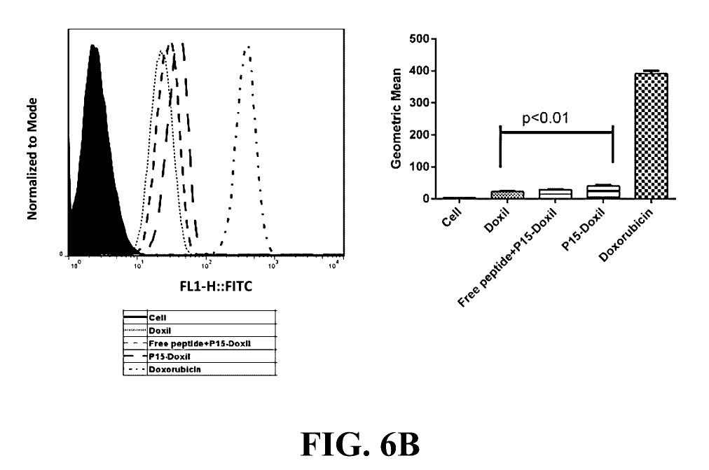

Referring to FIGS. 6A and 6B, the geometric mean fluorescence intensity (MFI) of doxorubicin in cells was analyzed by using flow cytometry (FCM) in the FL2 channel. The uptake of doxorubicin in both C26 and NIH3T3 cells is too high because doxorubicin can enter the cells by simple diffusion. Also uptake of Doxil, P15 Doxil and free peptide+P15 Doxil are the same in NIH3T3 cells, because the HDM-2 receptors are not overexpressed on them and they can enter the cell by same mechanism. However, uptake of P15-Doxil is more than uptake of Doxil because P15-Doxil is targeted and in C26 cells the HDM-2 receptors are overexpressed. Accordingly, a higher amount of doxorubicin can enter the C26 cells by P15-Doxil treatment. The specificity of P15-Doxil uptake was also evaluated by a competitive assay as described above.

### Example 6

### In Vivo Tumor-Targeted Therapeutic Studies

Animal care was carried out according to guidelines established by Institutional Ethical Committee and Research Advisory Committee of Mashhad University of Medical Sciences. Tumor models were C26 colon carcinoma in BALB/c mice Female BALB/c mice 4-6 week's old and female C57BL/6 mice 6-12 weeks old were injected subcutaneously in the right flank with tumor cells. Mice with size-matched tumors (approximately 10 mm³) were then randomly assigned to different treatments groups (7 groups with five mices) and were injected at 15 mg/kg or 10 mg/kg doxorubicin equivalent with either Doxil, P15-Doxil formulations and free doxorubicin or equivalent volumes of saline through the tail vein single dose.

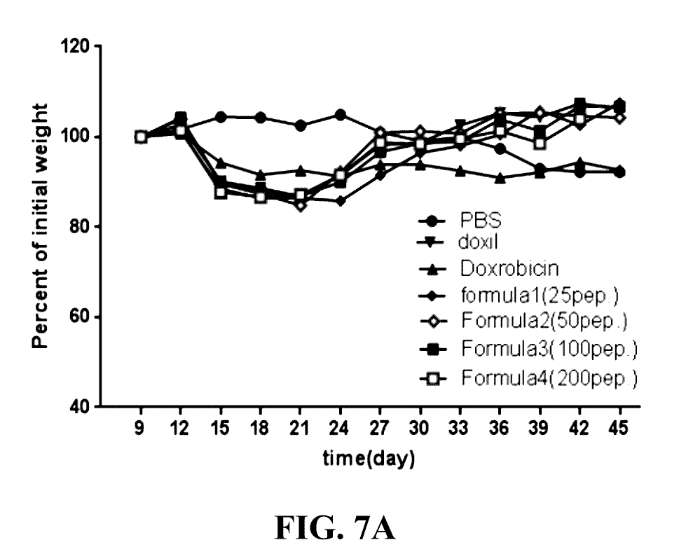

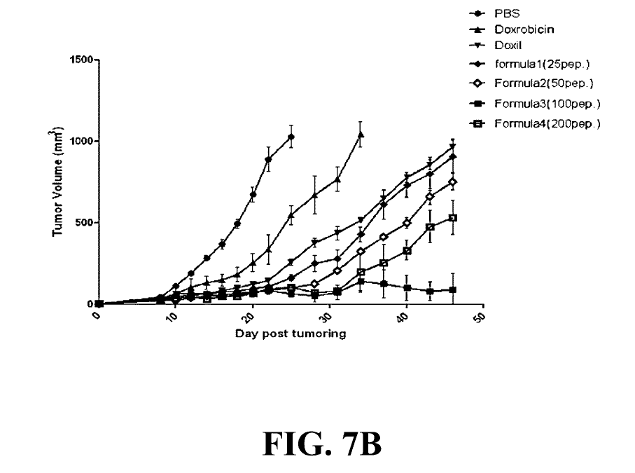

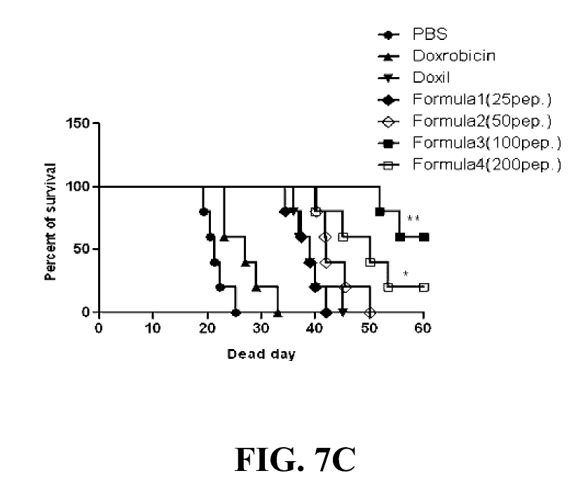

Mouse body weight was monitored three times a week for 60 days. Referring to FIG. 7A, mouse body weight according to percent of initial weight was reported and it can be understood that effect of P15-Doxil in body weight was approximately as same as Doxil effect and suggesting that the treatments did not produce any apparent toxicity

Also tumor size was monitored three times a week for 60 days. Three dimensions of tumor (height, length and width) were measured with calipers and tumor volume was calculated via the following formula: tumor volume=(height×length×width)×0.5 cm³. According to instructions for C26 tumor bearing animals, euthanasia was occurred when their body weight decreased (>20% loss of initial mass), their tumor was greater than 2.0 cm in one dimension or they became sick and unable to feed.

Referring to FIG. 7B, treatment with P15 (100 peptide)-Doxil, has a better effect in tumor volume and size of the tumor in this treatment is significantly smaller than other forms of doxorubicin treatments. Also according to FIG. 7C treatment with P15 (100 peptide)-Doxil shows higher percent of survival than other forms after 60 days.

The median survival time (MST) and time to reach end point (TTE) for each mouse were calculated from the equation of the line obtained by exponential regression of the tumor growth curve. Subsequently, the percent of tumor growth delay (TGD) were calculated based on the difference between the mean TTE of treatment group (T) and the mean TTE of the control group (C) (% TGD=[(T−C)/C]×100). Table 3 reports the results of MST, TTE and TGD of different treatments of doxorubicin; and it shows that the therapeutic efficacy of targeted P15-Doxil is better than Doxil and other forms of treatments.

TABLE 3
Therapeutic efficacy data of P15-Doxil different formulations,
Doxil, and free doxorubicin in mice bearing C-26 tumor.

| Treatment | MST (day) | TTE (days ± SD) | TGD (%) |
|---|---|---|---|
| Dextrose 5% | 21.3 | 21.7 | — |
| Doxorubicin 15 mg/kg | 27 | 27 | 24.42 |
| Doxil 15 mg/kg | 39 | 39.4 | 81.56 |
| P15 (25pep.)- | 39 | 38.55 | 77.65 |
| Doxil 15 mg/kg | | | |
| P15 (50pep.)- | 42 | 43.84 | 102 |
| Doxil 15 mg/kg | | | |
| P15 (100pep.)- | undefined | 57 | 162.67 |
| Doxil 15 mg/kg | | | |
| P15 (200pep.)- | 50 | 49.68 | 129 |
| Doxil 15 mg/kg | | | |

### Example 7

### Bio-Distribution Study

In this example, bio-distribution of drug was studied. For this purpose, two weeks after tumor inoculation, when the tumors were approximately 60-70 mm³, mice were injected with Doxil, P15-Doxil formulations and free doxorubicin or equivalent volumes of saline as previously mentioned. Blood samples were collected 3, 6, and 12 hours after drug injection via retro orbital bleeding.

Animals were sacrificed at 24 and 48 h after drug administration and blood samples were drawn by heart puncture. The whole tumor, one of the kidneys, spleen, heart, lungs as well as a portion of the liver and muscle were dissected, weighted and homogenized in acidified isopropanol with zirconia beads by Mini-Beadbeater-1 (Biospec, OK). Then the blood and the homogenized tissue samples were centrifuged and the blood serum as well as the sera of tissue samples were diluted in adequate volume of acidified isopropanol and stored at 4° C. overnight for Doxorubicin extraction.

The samples were centrifuged and the supernatant was assayed for Doxorubicin concentration by measuring fluorescence at λEx/Em=480/590 nm compared with Doxorubicin calibration curves, which were prepared in the tissue and sera extracts of the control mice. The pharmacokinetic concentration of doxorubicin was shown as μg/ml of serum. The samples were then centrifuged and the supernatant was assayed for Dox concentration spectro-fluorimetrically (Excitation wave length: 470 nm, Emission wave length: 590 nm). The calibration curve was prepared using serial dilutions of Dox in the tissue and sera extracts of the control mice.

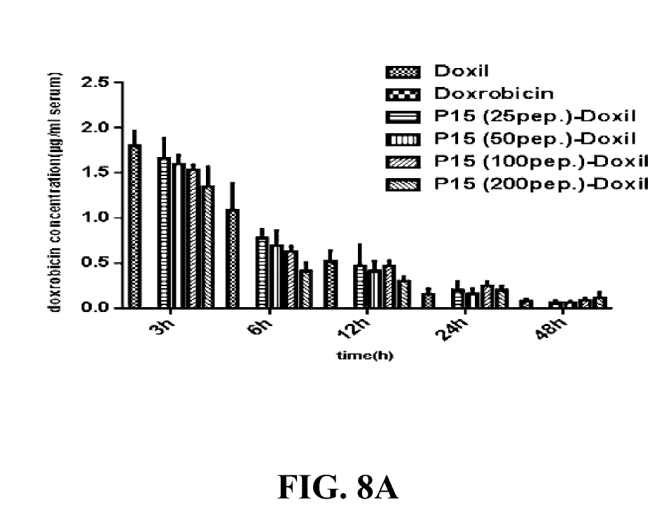

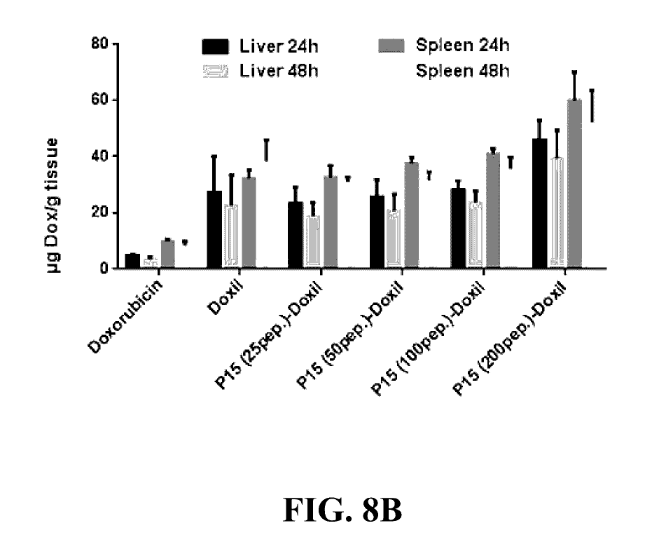

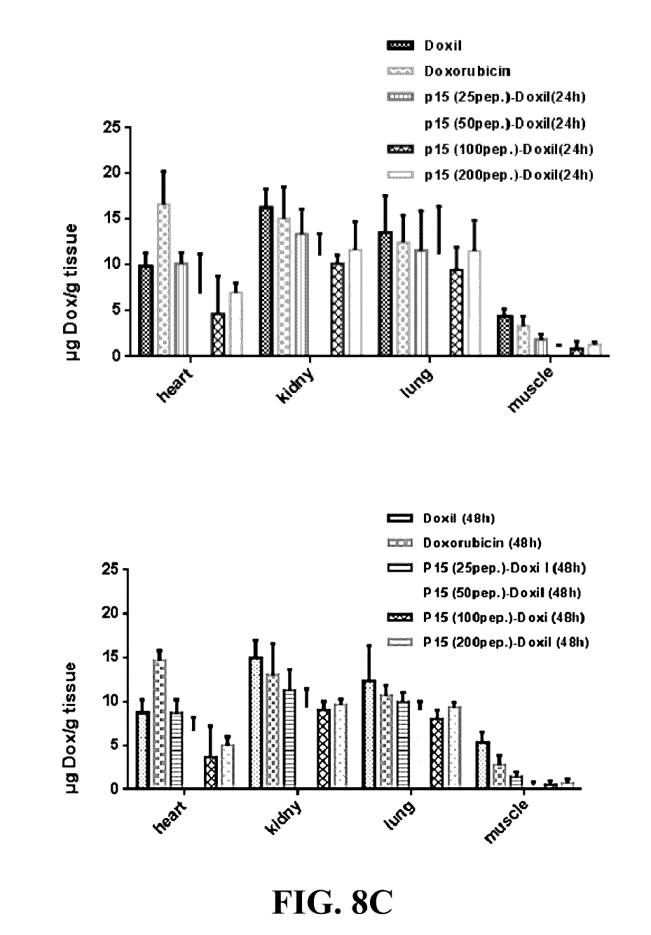

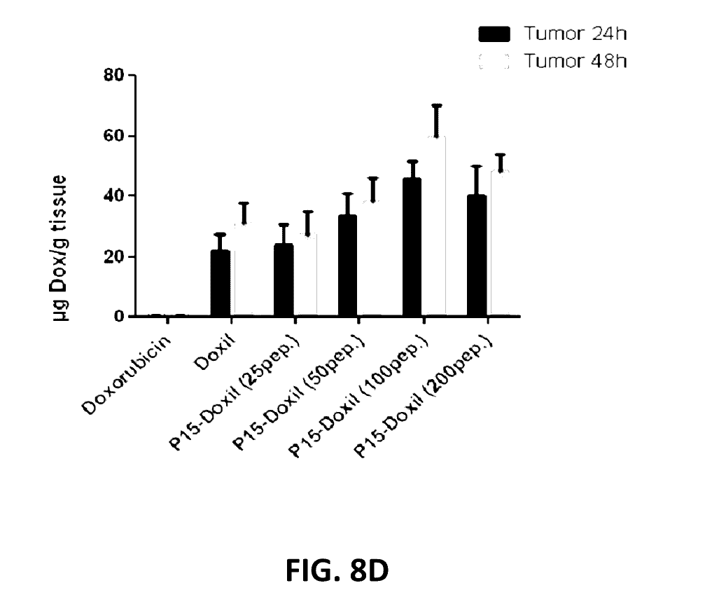

Referring to FIG. 8A, bio-distribution of P15-Doxil formulations, Doxil, and free doxorubicin at different time points (24 and 48 h) in serum was reported. It shows a decreasing trend over time.

According to FIG. 8B, bio-distribution of P15-Doxil formulations, Doxil, and free doxorubicin in liver and spleen in BALB/c mice bearing C-26 tumor after a single dose of 15 mg/kg liposomal doxorubicin administered intravenous two weeks after the tumor inoculation was reported. It shows that P15 (100 peptide) Doxil treatment is significantly better than treatment with P15 (200 peptide)-Doxil formulation; because it causes lower doxorubicin concentration in Reticuloendothelial System (RES)-riche organs such as liver and spleen after treatment.

Referring to FIG. 8C, bio-distribution of P15-Doxil formulations, Doxil, and free doxorubicin are illustrated at different time points (24 and 48 h) in different organs including kidneys, lungs, muscle and heart of BALB/c mice bearing C-26 tumor after intravenous administration of a single dose of 15 mg/kg liposomal doxorubicin, two weeks after the tumor inoculation and it shows less drug accumulation of P15-Doxil formulations compared to Doxil in different organs 24 and 48 hours after treatment. Data showed as mean±S.E.M. (n=3)

Bio-distribution of P15-Doxil formulations, Doxil, and free doxorubicin at different time points (24 and 48 h) in tumor in BALB/c mice bearing C-26 tumor after a single dose of 15 mg/kg liposomal doxorubicin administered intravenous two weeks after the tumor inoculation was shown in FIG. 8D. Referring to FIG. 8D, treatment with P15 (100 peptide)-Doxil cause the most concentration of drug in tumor 24 and 48 hour after treatment; so the P15 (100 peptide)-Doxil is the best formulation for tumor (cancer) treatment. Data expressed as mean±S.E.M. (n=3).

The P15-liposomes as described in this disclosure can target HDM-2 receptors on tumor cells and the aforementioned liposomes may be utilized to deliver anticancer drugs (e.g., doxorubicin, paclitaxel, etc.) to HDM-2 expressing cancer cells. Therefore, in an aspect, a method for treatment of cancer using the liposomal composition of the present disclosure may include administering a therapeutically effective amount of the P15-liposomes loaded with an anticancer drug to a human or animal in need thereof. The term “therapeutically effective amount” is an amount that is effective to ameliorate a symptom of a disease.

What is claimed is:

1. A liposomal composition for treatment of cancer, comprising:
a targeted PEGylated liposome, wherein the targeted PEGylated liposome is targeted by P15 molecules ranging from 25 to 100, wherein the P15 molecules are P15 peptide with a sequence of H-Cys-Gly-Gly-Gly-Pro-Pro-Leu-Ser-Gln-Glu-Thr-Phe-Ser-Asp-Leu-Trp-Lys-Leu-Leu-OH, and
wherein the targeted PEGylated liposome is loaded with doxorubicin.

2. A liposomal composition for treatment of cancer, comprising:
a targeted PEGylated liposome, wherein the targeted PEGylated liposome is targeted by an effective number of P15 molecules,
wherein the targeted PEGylated liposome is loaded with an anticancer drug.

3. The liposomal composition according to claim 2, wherein the P15 molecules are P15 peptide with a sequence of H-Cys-Gly-Gly-Gly-Pro-Pro-Leu-Ser-Gln-Glu-Thr-Phe-Ser-Asp-Leu-Trp-Lys-Leu-Leu-OH.

4. The liposomal composition according to claim 2, wherein the anticancer drug is selected from the group consisting of Epigallocatechin-3-gallate (EGCG), soy isoflavones, Isoflavones genistein, daidzein, Coumarins, flavonoids, silibinin, polyphenols, baicalin, lycopenes, Vincristine, doxorubicin, cisplatin, 5-fluorouracil, Methotrexate, Cyclophosphamide, mustine, prednisolone, epirubicin, folinic acid, oxaliplatin, etoposide, bleomycin, and combinations thereof.

5. The liposomal composition according to claim 2, wherein the anticancer drug is selected from the group consisting of doxorubicin, daunorubicin, epirubicin, Idarubicin and derivatives thereof.

6. The liposomal composition according to claim 2, wherein the anticancer drug is doxorubicin.

7. The liposomal composition according to claim 2, wherein the P15 molecules bind at least one extracellular domain of an HDM-2 receptor.

8. The liposomal composition according to claim 2, wherein the effective number of peptide molecules is between 25 and 200.

9. The liposomal composition according to claim 2, wherein the effective number of peptide molecules on the surface of said PEGylated liposome is 100.

10. A method for preparing targeted PEGylated liposomes, the method comprising:
preparing phospholipid-PEG2000-P15 micelles; and
post-inserting the phospholipid-PEG2000-P15 micelles into preformed liposomes to obtain targeted liposomes.

11. The method according to claim 10, wherein the phospholipid is 1,2-Distearoyl-sn-glycero-3-phosphoethanolamine (DSPE).

12. The method according to claim 10, wherein preparing phospholipid-PEG2000-P15 micelles includes conjugating the P15 peptide with a DSPE-PEG2000 maleimide.

13. The method according to claim 10, wherein post-inserting the phospholipid-PEG2000-P15 micelles into the preformed liposome includes co-incubating the DSPE-PEG2000-P15 micelles and the preformed liposomes at 60° C. under a gentle shaking.

14. The method according to claim 10, further comprising purifying the obtained targeted liposome using dialysis against Histidin buffered with 30 kDa molecular weight cut off (MWCO) dialysis membrane.
